(19) United States
(12) Patent Application Publication
(10) Pub. No.: US 2014/0303451 A1
(43) Pub. Date: Oct. 9, 2014

(54) Health Monitoring Apparatus

(71) Applicants: John L. Beiswenger, Strasburg, PA (US); James A. Wilson, Lancaster, PA (US)

(72) Inventors: John L. Beiswenger, Strasburg, PA (US); James A. Wilson, Lancaster, PA (US)

(21) Appl. No.: 13/859,328

(22) Filed: Apr. 9, 2013

(51) Int. Cl. A61B 5/087; A61B 5/024; A61B 5/01

(52) U.S. Cl. CPC A61B 5/087; A61B 5/01; A61B 5/02416
USPC 600/301

(57) ABSTRACT

A health monitoring apparatus is providing, which includes a body section and a housing section that covers the body section. The body section includes a printed circuit board, a flow meter section, a first mouth probe, a second mouth probe, and a non-contact thermometer light guide. The flow meter section includes a base and a pair of channel walls extending upward from the base and a gap there between to define a fluid flow channel having a fluid receiving passageway. The first mouth probe extends outward from the fluid flow channel, and the second mouth probe is disposed axially with respect to the first mouth probe and extends outward from the fluid flow channel. The non-contact thermometer light guide is disposed along the first mouth probe and leads into the flow meter section. The non-contact thermometer light guide includes an electromagnetic radiation sensor disposed along a trailing end of the non-contact thermometer light guide.

### FIELD OF THE INVENTION

The present invention is related to a health monitoring apparatus and, more particularly, to a health monitoring apparatus and system that predicts and alerts a pre-symptomatic health change due to an impending infection

### BACKGROUND

Individuals are susceptible to respiratory infections including influenza, bronchitis and pneumonia. In the United States alone, ordinary pneumonia killed 62,000 people in 1999. Asthma and chronic obstructive pulmonary disease (COPD) patients are particularly susceptible to respiratory infections. Approximately seventeen million people are known to have asthma in the United States, and approximately twelve million adults have COPD. It is estimated that approximately another twelve million people may have COPD, but do not know it.

Infectious disease is the third leading cause of death in the United States. Moreover, deaths from infectious disease have been increasing. More Americans died per thousand due to infectious disease in 2000 than in 1980. Approximately, 90,000 people die each year in the United States due to nosocomial infections, many resulting from surgery. Individuals whose immune systems are compromised are particularly susceptible.

Peak flow meters are frequently recommended to monitor the lung function value of asthmatics' airways. These devices provide a non-invasive means to monitor lung function values and can be used at home by asthmatics of all ages. Peak flow er readings fall before symptoms of asthma are otherwise noticed suggesting an impending respiratory episode (providing early detection).

Primary Prevention (i.e., taking steps to prevent the occurrence of an infection) is, of course, important. However, sequestration or quarantine is of value only in epidemic situations, and because of social activity, infections are still transmitted.

Secondary Prevention is the early detection of an infection followed by actions taken immediately to reverse, halt or retard its progression (e.g., taking antiviral drugs, anti-oxidants or antibiotics). Secondary Prevention can be very effective in protecting individuals from the adverse effects of infectious disease, but most therapeutic treatments are most effective when started during the early stages of an infectious disease. Furthermore, detecting infections in their very early stages has been difficult since physical symptoms are often absent.

Metabolism is defined as the entire range of biochemical processes that occur within the body. Metabolic rate is defined as the amount of energy released or stored through biochemical processes. Metabolic rate is measured in the amount of heat released per unit of time. Body heat is produced primarily by metabolic processes including muscular activity.

Body temperature must be maintained in a narrow range for survival and is influenced by metabolic rate. Body temperature is controlled by the body's thermoregulatory system which includes the hypothalamus. The resulting average daytime body temperature in adults (taken near the sublingual artery) is 98.2 degrees plus or minus 1 degree.

Basal metabolic rate (BMR) is the measure of the lowest level of metabolism that occurs in a normal 24-hour period. BMR occurs mid-sleep cycle when an individual is at digestive, physical and emotional rest. Body temperature drops to its lowest point corresponding to BMR (defining basal temperature, Tb).

Tb can be monitored with temperature taken immediately upon waking, the waking temperature (Tw). Hormones, such as progesterone and estrogen, modulate metabolic rate and therefore Tw. With the release of hormones, cyclical Tw readings result, requiring the use of properly scaled graphs to generate accurate baselines.

When infectious agents invade body tissues, leukocytes and macrophages are mobilized to fight the infection. These infection-fighting biochemical processes increase metabolic rate, causing an increase in Tw. An increase in Tw of as little as 0.5 degrees above an established baseline may indicate an infection is present. Monitoring Tw can be used for early detection of infection. Tw is a pre-symptomatic sign.

Peak expiratory flow (PEF) readings fall before respiratory symptoms are apparent. PEF readings are lower upon waking, which is the waking peak flow (PFW). If PFW readings drop below baseline levels, maintained on a properly scaled graph, a respiratory event may be imminent.

Oxygen saturation refers to the extent to which hemoglobin is saturated with oxygen. Hemoglobin is an element in the blood that binds with oxygen to carry it through the bloodstream to the organs, tissues and cells of the body. It is generally known that a reduction in blood oxygen saturation in COPD patients is an indication of a worsening condition.

Fractional exhaled oxygen (FEO₂) and fractional exhaled nitric oxide (FENO) are both important indications of respiratory health.

Furthermore, it is generally known that an individual's heart rate (HR) increases with fever and infection.

Therefore, it is important to monitor the pre-symptomatic signs such as Tw, PFW, and HR, among other factors, when effectively predicting health changes. As a result, a need exists for an effective means for detecting and reporting early impending respiratory and non-respiratory infections.

### SUMMARY

In view of the aforementioned shortcomings, a health monitoring apparatus and a system for monitoring pre-symptomatic health change signs according to the invention is provided.

The health monitoring apparatus includes a body section and a housing section that covers the body section. The body section includes a printed circuit board, a flow meter section, a first mouth probe, a second mouth probe, and a non-contact thermometer light guide. The flow meter section includes a base and a pair of channel walls extending upward from the base and a gap there between to define a fluid flow channel having a fluid receiving passageway. The first mouth probe extends outward from the fluid flow channel, and the second mouth probe is disposed axially with respect to the first mouth probe and extends outward from the fluid flow channel. The non-contact the thermometer light guide is disposed along the first mouth probe and leads into the flow meter section. The non-contact thermometer light guide includes an electromagnetic radiation sensor disposed along a trailing end of the non-contact thermometer light guide.

### BRIEF DESCRIPTION OF THE DRAWINGS

Exemplary embodiments of the invention will be described with reference to the accompanying drawings, of which:

FIG. 1 is a top left perspective view of a health monitoring apparatus according to the invention;

FIG. 2 is a rear exploded perspective view of the health monitoring apparatus according to the invention;

FIG. 3 is a bottom perspective of the health monitoring apparatus according to the invention, showing a display unit of the health monitoring apparatus according to the invention;

FIG. 4 is a diagram of the display unit, showing an exemplary illustration of an indication of a probable infection, respiratory episode and/or respiratory infection as detected by the health monitoring apparatus according to the invention.

FIG. 5 is a partial exploded perspective view of the health monitoring apparatus according to the invention, showing a mouthpiece section leading into a partial view of a flow meter section according to the invention;

FIG. 6 is a section view of the health monitoring apparatus shown in FIG. 5, taken along line 6-6;

FIG. 7 is another section view of the health monitoring apparatus shown in FIG. 5, taken along line 7-7;

FIG. 8 is another section view of the health monitoring apparatus shown in FIG. 5, taken along line 8-8;

FIG. 9 is a top right perspective view of another health monitoring apparatus according to the invention, having a fractional exhaled oxygen (FEO₂) detector and a fractional exhaled nitric oxide (FENO) detector positioned in the flow meter section according to the invention;

FIG. 10 is a diagram showing an exemplary position of use for the health monitoring apparatus according to the exemplary embodiments of the invention; and

FIG. 11 is an illustration of a known anatomical view of the vertical muscle of the tongue and the location of the right sublingual artery.

### DETAILED DESCRIPTION OF THE EMBODIMENT(S)

Now with reference to the Figures, a health monitoring apparatus 1 according to the invention, and a system for monitoring individuals and/or populations to predict impending changes in health and provide alerts of those changes will be described.

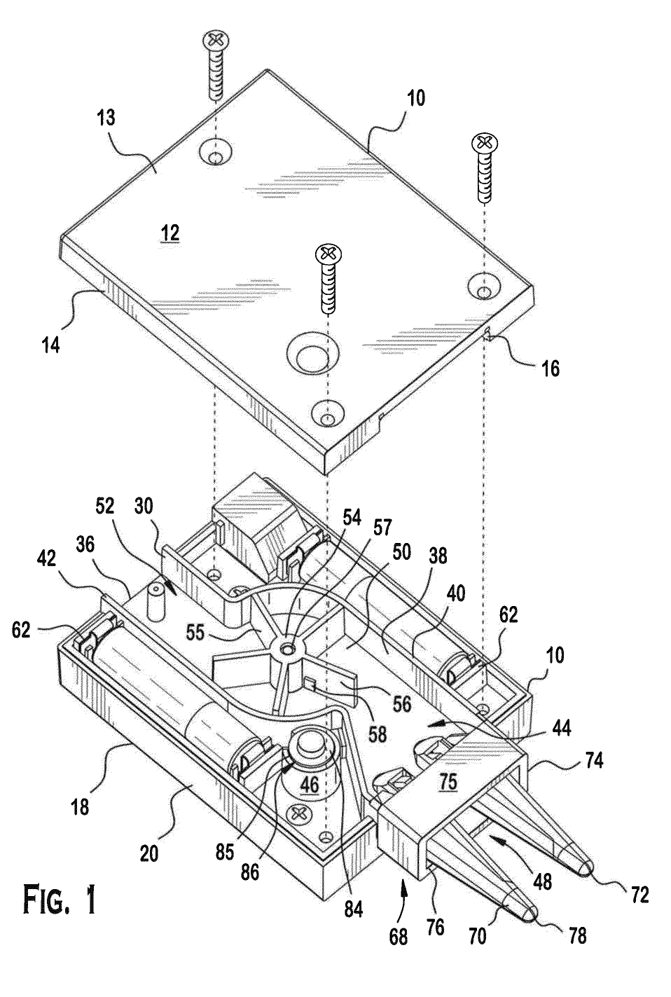

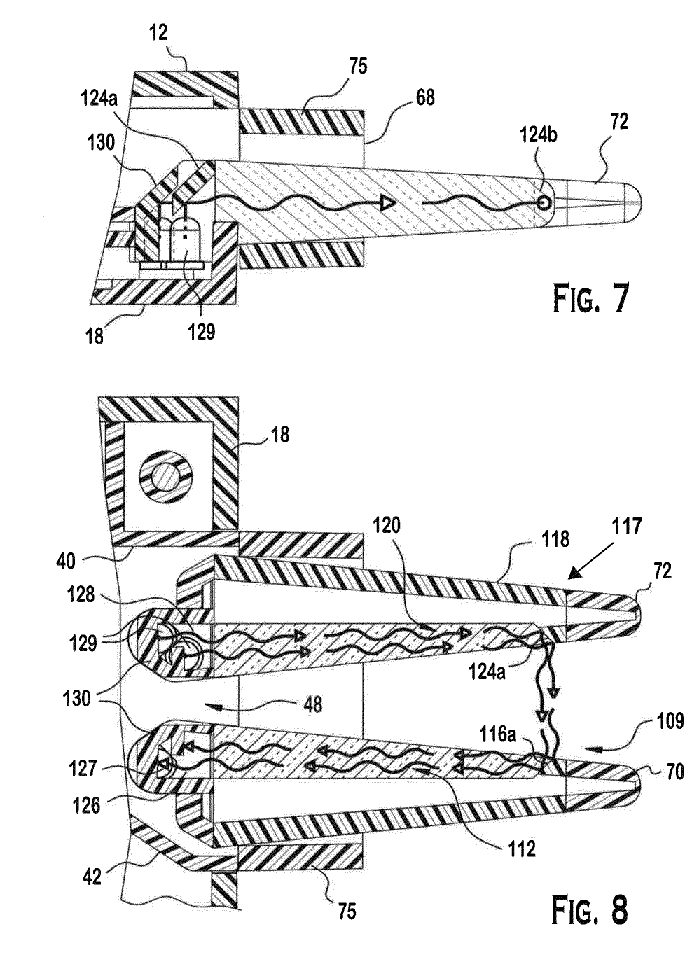

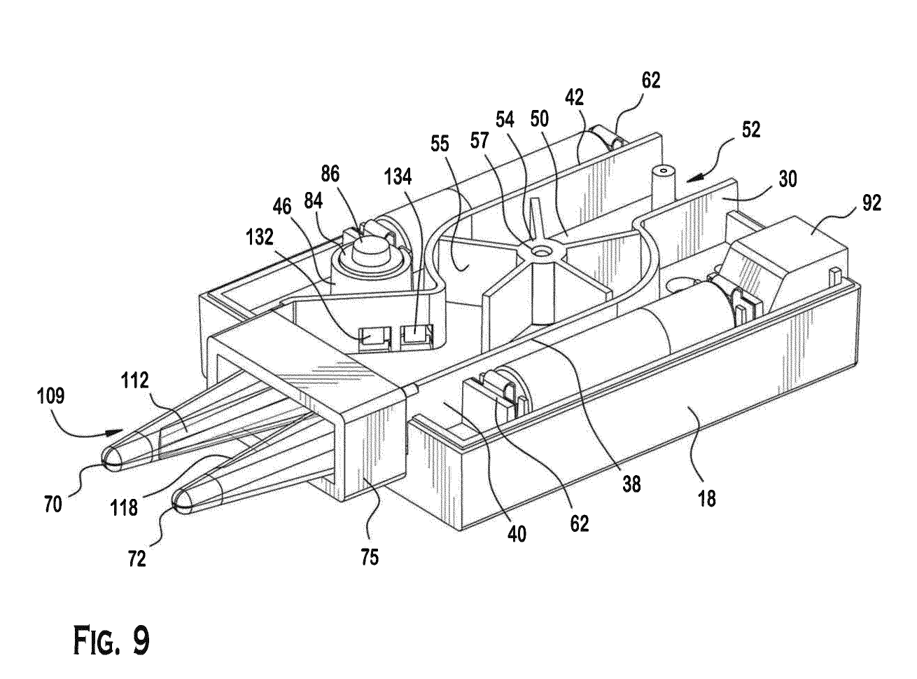

In general, as shown in FIGS. 1-9, the health monitoring apparatus 1 includes the following major components: a body section 30, a housing section 10, and a cover 24.

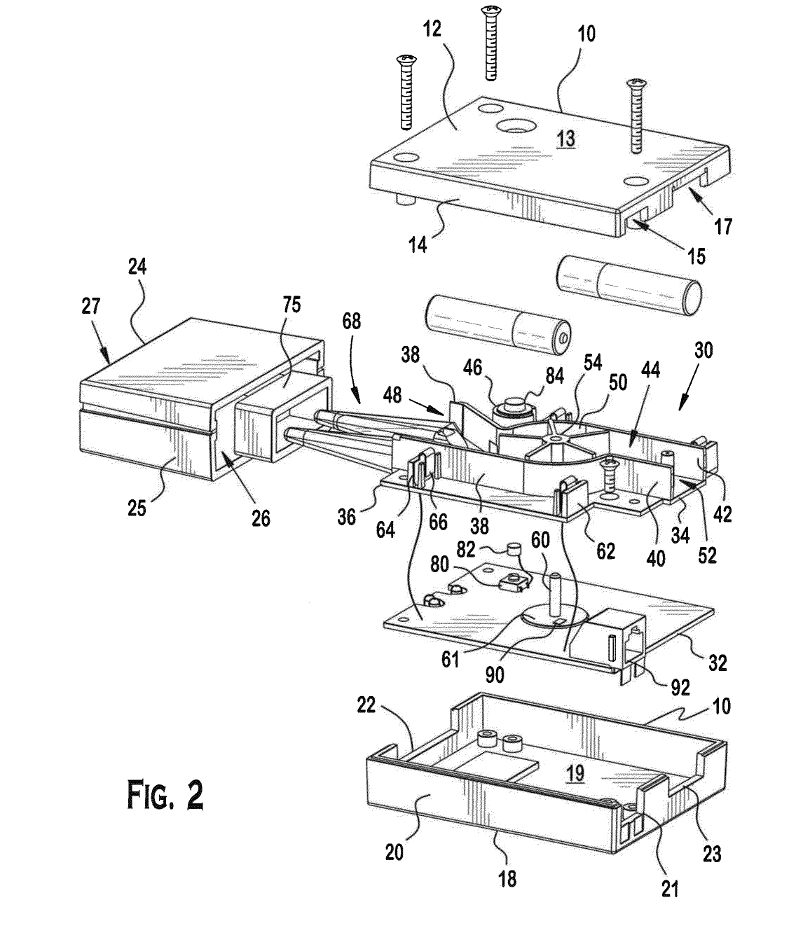

With reference to FIGS. 1 and 2, an exemplary embodiment of the body section 30 is shown and includes a printed circuit board (PCB) 32, a flow meter section 34, and a mouthpiece section 68.

As shown in FIG. 2, the PCB 32 is planar, non-conductive substrate having a plurality of conductive pathways, such as tracks or signal traces, etched from copper sheets laminated onto the non-conductive substrate. An integrated circuit central processing unit (CPU IC) is disposed on a lower surface of the PCB 32 and is electrically connected to the traces. A memory unit is provided and positioned proximate to the CPU IC in the embodiment shown. The memory unit is electrically connected to the CPU IC through the conductive pathways.

As further shown in FIGS. 1 and 2, a communication device 92 is also provided and, in the embodiment shown, positioned on the PCB 32. In particular, the communication device 92 is a modular connector socket, such as a telephone or data jack that is electrically connected to the CPU IC through the conductive pathways. In the embodiment shown, the communication device 92 is disposed on a top surface of the PCB 32. However, depending on the type of communication port used, the communication device 92 could be positioned differently than shown. One skilled in the art should appreciate that other communication ports may be used, including a serial bus interfaces, such as a universal serial bus (USB) connectors, micro USB, IEEE 1394 connector, wireless data exchange device, such as Bluetooth® technology or WiFi technology, or other communications connectors known to one skilled in art.

Now with reference to FIGS. 1 and 2, the flow meter section 34 is adjacently positioned to the PCB 32 to minimize space in the health monitoring apparatus 1. The flow meter section 34 generally includes a base 36 and a plurality of channel walls 38 extending upward from a top surface of the base 36.

As shown in FIG. 2, the base 36 is a thin planar member having a substantially rectangular shape with uniform thickness throughout. As shown, the base 36 includes a plurality of notches along a perimeter of the base 36, such that components disposed on the upper surface of the PCB 32 can extend there through. Additionally, in the embodiment shown, the base 36 includes a plurality of fastener passageways that extend through the base 36 and are dimensioned to receive a fastener there through. One skilled in the art should appreciate that the shape and thickness of base 36 may have varied shape and size, depending on design requirements of the health monitoring apparatus 1 and the number of data collecting components required for a particular purpose thereof.

In the embodiment shown, the base 36 is manufactured from a molded plastic. However, one skilled in the art should appreciate that the base 36 can be prepared from other medical materials, such as metals, ceramics, and/or composites.

In the shown embodiment, each channel wall 38 is positioned substantially orthogonal with respect to the top surface of the base 36. Furthermore, each channel wall 38 has a uniform height in the embodiment shown. Each channel wall 38 wholly extends from a front side of the base 36 to a rear side of the base 36 with a gap provided there between the pair of channel walls 38, which provides a fluid flow channel 44 positioned between the pair of channel walls 38.

In the embodiment shown, the fluid flow channel 44 is dimensioned by the pair of channel walls 38 and the base 36. Each channel wall 38 is a side wall for the fluid flow channel 44, while the base 36 serves as a floor of the fluid flow channel 44 in the embodiment shown. However, one skilled in the art should appreciate other design configurations are possible.

In the shown embodiment, a first channel wall 40 and a second channel wall 42 are sidewalls for the fluid flow channel 44.

In particular, as shown in FIG. 1, the first channel wall 40 first extends from the front side linearly toward a substantial central section of the base 36, and then curves toward a second channel wall 42 defining a semi-circle shape in the embodiment shown. Then, the first channel wall 40 bends back toward the rear side, at a substantial right angle in the embodiment shown, and extends linearly to an edge of the rear side. However, one skilled in the art should appreciate that the first channel wall 40 can be designed in various configurations, having more or less bends and arcs, such as along as the deviations from the exemplary embodiment does not depart from the spirit and function of other components of the invention.

Also shown in FIG. 1, the second channel wall 42 extends from the front of the base 36 toward the rear. However, unlike the first channel wall 40, the second channel wall 42 initially extends toward the center section of the base 36 at a modest incline. In fact, in the embodiment shown, the second channel wall 42 extends toward the first channel wall 40 at a 45 degree angle. As a result, the fluid flow channel 44 initially narrows, with a gap between the first channel wall 40 and the second channel wall 42 narrowing, until it reaches the center section of the base 36. Then, the second channel wall 42 curves away from the first channel wall 40. As shown, the second channel wall 42 follows the curvature of the first channel wall 40 to provide a circular section of channel walls 38 proximate the center section of the base 36. Then, the second channel wall 42 extends back toward the rear side of the base 36, such that first and second channel walls 40, 42 extend parallel to each other, until both the first and second channel wall 40, 42 reach the rear of the base 36. However, this is just one possible configuration of the channel walls 38, and the fluid flow channel 44 may have other designs. One skilled in the art should appreciate the channel walls 38 could be positioned in various configurations, having more or less bends and arcs, such as along as the deviations from the exemplary embodiment does not depart from the spirit and function of the health monitoring apparatus 1 according to the invention.

As shown in FIG. 1, the fluid flow channel 44 includes a rotor receiving section 50, which is a receiving space that is positioned between the first channel wall 40 and the second channel wall 42. In particular, as shown, the rotor receiving section 50 is positioned between the curved sections of the first and second channel walls 40, 42. As a result, the rotor receiving section 50 is circular shaped in the embodiment shown, with each channel wall 38 mirroring the other around a proximate central section of the base 36.

As shown in FIG. 1, a fluid receiving passageway 48 is provided in addition to the fluid flow channel 44. The fluid receiving passageway 48 is positioned along the front edge of the base 36. The fluid receiving passageway 48 is an opening along the front of the body section 30, which leads into the fluid flow channel 44. The fluid receiving passageway 48 is positioned between the pair of channel walls 38 and above the base 36.

Additionally, as shown in FIG. 1, a fluid exhaust passageway 52 is provided along a rear end of the fluid flow channel 44 and positioned opposite the fluid receiving passageway 48. Like the fluid receiving passageway 48, the fluid exhaust passageway 52 is an opening positioned between the pair of channel walls 38. However, the fluid exhaust passageway is disposed along the rear side of the base 36 in the embodiment shown. The fluid flow channel 44 exits through the fluid exhaust passageway 52 in the embodiment shown. However, one skilled in the art should appreciate that other design configurations are possible. For instance, the first and second channel walls 40, 42 may be configured to exit along another side of the fluid meter section 34.

As shown in FIG. 1, the flow meter section 34 further includes a sensor receiving wall 46 extending upward from the base 36. In the embodiment shown, the sensor receiving wall 46 is a wall or plurality of walls extending upward from the base 36 and positioned adjacent to the fluid flow channel 44. In particular, the sensor receiving wall 46 is positioned outside the fluid flow channel 44 and adjacent the second channel wall 42. The sensor receiving wall 46 is a circular wall in the embodiment shown, and includes a receiving passageway that extends through and out a bottom surface of the base 36. The sensor receiving wall 46 has a height equal to a height of the pair of channel wails 38. However, one skilled in the art should appreciate that the shape and position of the sensor wall are merely exemplary and that the sensor wall could be shaped and positioned in a variety of designs.

As shown in FIGS. 1 and 2, the body section 30 also includes a power source, such as a battery. Accordingly, the body section 30 includes a battery receiving section 62 positioned on a surface of the base 36 in the embodiment shown. In particular, the battery receiving section 62 is positioned adjacent and outside of the channel wails 38. The battery receiving section 62 includes a pair of opposing retaining walls 64 that face each other. Each opposing retaining wall 64 includes a metal plate 66 along inner surfaces thereof, which is electrically connected to the PCB 32 using the conductive pathways, in the embodiment shown. Each battery receiving section 62 is dimensioned such that power source, such as an AAA or other type of battery can fit there between, and that the terminals of the battery contact each of the respective metal plates 66 of the pair of opposing retaining walls 64.

In the shown embodiment, a pair of battery receiving sections 62 are positioned on opposite side surfaces of the base 36. Each battery receiving section 62 is dimensioned to receive a AAA battery, in the embodiment shown. However, one skilled in the art should appreciate that the placement, shape, and dimensions of the battery receiving section 62 may change depending on the type of power source used. For instance, a button cell battery may be used, which would require different structure, such as a circular retaining wall(s) that receives and secures the button cell battery therein, and metal plates 66 would be positioned to appropriately contact terminals of the button cell battery.

In another alternative embodiment, it is possible to power the health monitoring apparatus 1 using a rechargeable battery, or a direct power source, such that a power outlet is positioned on the body section 30. Various combinations of power sources can be used, and the battery receiving section 62 can be modified to accommodate the type of power source selected in powering the health monitoring apparatus 1.

As shown in FIGS. 1 and 2, the mouthpiece section 68 is positioned on one side of the flow meter section 34, and extends away there from. In particular, the mouthpiece section 68 extends laterally outward from a front side of the base 36 and includes a first mouth probe 70 and a second mouth probe 72 in the shown embodiment.

The first mouth probe 70 is an elongated member having a trailing end positioned through the fluid receiving passageway 48 and secured to an interior upper surface of the base 36. In the embodiment shown, the trailing end of the first mouth probe 70 is firmly, integrally secured to the base 36 in the fluid flow channel 44, but could otherwise be removably attached to the base 36. In the alternative, the trailing end of the first mouth probe 70 could be integrally secured or removably attached there to the first mouth probe 70. However, in the embodiment shown, the trailing end is positioned adjacent the second channel wall 42 and then extends outward from the base 36, through the fluid receiving passageway 48, and tapers to a leading end having a blunt tip.

The second mouth probe 72 is also an elongated member having generally the same shape and dimensions as the first mouth probe 70. The second mouth probe 72 includes a trailing end positioned through the fluid receiving passageway 48 and secured to the base 36 in the fluid flow channel 44. However, the trailing end is positioned adjacent the second channel wall 42, and opposite the first mouth probe 70 in the fluid flow channel 44. In the embodiment shown, the trailing end of the second mouth probe 72 is firmly, integrally secured to the base 36, but could otherwise be removably attached to the base 36. Additionally, the second mouth probe 72 could be integrally secured with or removably attached to the first channel wall 40 in other exemplary embodiments. The second mouth probe 72 then extends outward from the base 36, through the fluid receiving passageway 48, and tapers to a leading end in the shown embodiment. A gap is provided laterally between the first mouth probe 70 and the second mouth probe 72, such that an opening is provided into the fluid receiving passageway 48, and the fluid flow channel 44 is unobstructed.

As shown in FIGS. 1 and 2, the mouthpiece section 68 further includes a mouthpiece housing 74 having outer walls 75 and a mouth probe receiving passageway 76 extending between the outer wails 75. The mouthpiece section 68 is dimensioned such that the outer walls 75 wrap around each of the first and second mouth probes 70, 72 and sit flush with the front edge of the base 36, as well as the leading ends of the first and second channel walls 40, 42. The mouth probe receiving passageway 76 includes a fluid passageway that is dimensioned to receive both the first and second mouth probes 70, 72, but still includes an opening that leads into the fluid receiving passageway 48 and fluid flow channel 44. In the embodiment shown, an inner edge of the outer walls 75 are removably attached to the flow meter section 34 and the first and second mouth probes 70, 72, but could otherwise be rigidly attached or integrally formed with the flow meter section 34.

Now with reference FIGS. 1 and 2, the housing section 10 generally includes an upper housing 12 and a lower housing 18.

In the shown embodiment, the upper housing 12 includes a rectangular planar support 13 with outer walls 14 extending downward from edges of the planar support 13. The upper housing 12 further includes a plurality of fastener receiving through holes in the embodiment shown. Additionally, the upper housing 12 includes a plurality of notches or openings disposed along the outer walls 14. Each notch or opening is dimensioned to provide access to internal components of the body section 30. In the embodiment shown, each notch and opening may be a communications device receiving passageway 15, a fluid entrance passageway 16, or a fluid exhaust passageway 17.

The lower housing 18 also includes a rectangular planar support 19 with outer walls 20 extending downward from edges of the planar support 19. The lower housing 18 is dimensioned to correspond with the upper housing 12. The lower housing 18 further includes a plurality of fastener receiving threaded passageways in the embodiment shown, which correspond in placement with the plurality of fastener receiving through holes in the upper housing 12. in addition, the lower housing 18 includes a plurality of notches or openings disposed along the outer walls 20. Each notch or opening is dimensioned to provide access to internal components of the body section 30. These notches and openings include a communications device receiving passageway 21, a fluid entrance passageway 22, and a fluid exhaust passageway 23, in the embodiment shown.

Both the upper housing 12 and the lower housing 18 are dimensioned such that the upper and lower housing 12, 18 receive and surround the body section 30. Additionally, the communications device receiving passageway 15, the fluid entrance passageway 16, and the fluid exhaust passageway 17 of the upper housing 12 respectively correspond with the communications device receiving passageway 21, the fluid entrance passageway 22, and the fluid exhaust passageway 23 of the lower housing 18.

As shown in FIG. 2, the cover 24 is a shell having outer walls 25, a mouthpiece receiving space 26, and a closed end 27. The cover 24 is dimensioned to fit over the mouthpiece section 68, including the mouthpiece housing 74.

The health monitoring apparatus 1 is structured to support one or more data collecting components, and are shown in each of the exemplary embodiments shown in FIG. 1-7. One skilled in the art should appreciate that the health monitoring apparatus 1 could include any of the discussed data collecting components, in different combinations, and that the exemplary embodiments shows just a few possible combinations, and others are possible depending on what data is required for the health monitoring apparatus 1 according to the invention.

Firstly, the embodiment shown in FIGS. 1 and 2 illustrates a rotor 55 positioned in the body section 30. In particular, the rotor 55 is positioned between the pair of channel walls 38 in the rotor receiving section 50. The rotor 55 is positioned in a direct path of the fluid flow channel 44. In the embodiment shown, the rotor 55 rests above the upper surface of the base 36.

The rotor 55 includes a hub 54 and a plurality of rotor blades 56 extending outward from the hub 54. The hub 54 is a cylindrical member having an axle receiving passageway 57 extending there through in the embodiment shown. Additionally, the rotor 55 in the embodiment shown includes a magnet or magnetic device 58 embedded therein. In particular, the magnet is isolated to one side of the hub 54. However, one skilled in the art would appreciate that the magnet or magnetic device 58 could be positioned in one or more of the rotor blades 56, as well.

Accordingly, in the shown embodiment, an axle 60 is provided that extends from a axle base 61. The axle 60 is a rod like elongated member dimensioned to fit in the axle receiving passageway 57 of the hub 54. The axle base 61 is a circular planar member that sits flush between the lower surface of the base 36 and the upper surface of the PCB 32 in the embodiment shown.

Additionally, as shown in FIG. 2, the body section 30 includes a rotor movement detector 90, such as field-effect transistor or other sensor, which is positioned on the PCB 32 and electrically connected to the CPU IC. In the shown embodiment, the rotor movement detector 90 is positioned on an upper surface side of the PCB 32 and corresponds with a travel path of the magnet or magnetic device 58 of the rotor 55, when the rotor 55 rotates in the rotor receiving section 50. However, one skilled in the art should appreciate that the placement of the rotor movement detector 90 can be modified according to the type of device used and that devices ability to measure the number of rotations of the rotor 55 or passes of the magnetic device 58. In the embodiment shown, the rotor movement detector 90 is integrally formed with the axle base 61, so that it corresponds with positioning and movement of the rotor 55.

As shown in FIG. 1, a thermometer tip 78 is disposed in the mouthpiece section 68. More particularly, the thermometer tip 78 is positioned along a tip of first mouth probe 70. In the shown embodiment, the thermometer tip 78 is a rapid electronic thermometer probe in the embodiment shown, and is positioned at the leading end of the first mouth probe 70. However, one skilled in the art should appreciate that the thermometer tip 78 could be positioned on the second mouth probe 72 in the embodiment shown, or both probes 70, 72 in other embodiments. The thermometer tip 78 is mounted on the tip of the first mouth probe 70 and then electrically connected to the CPU IC in the embodiment shown, using an electrical wire that runs from the first mouth probe 70 to the PCB 32.

As shown in the Figures, a power switch 80 is disposed on the PCB 32 and is electrically connected to the power source in the embodiment shown. The power switch 80 is positioned to correspond in placement with the sensor receiving wall 46. The power switch 80 includes a control mechanism, such as a button, that extends into the receiving passageway between the sensor receiving wall 46.

Additionally, a monitoring sensor 82 is positioned adjacent to the power switch 80, and in the embodiment shown the monitoring sensor 82 is positioned above the control mechanism of the power switch 80. In the shown embodiment, the monitoring sensor 82 is an IR sensor capable of determining heart rate. The monitoring sensor 82 may include a separate microprocessor or may be connected to the CPU IC. In the embodiment shown, the monitoring sensor 82 is also capable of collecting other health parameters from a human finger.

As shown in FIGS. 1 and 2, a sensor housing 84 is provided. The sensor housing 84 is a plastic housing having outer walls 85 and a spring member 86 positioned below the outer walls 85. In another embodiment, the sensor housing may be a resilient flexible rubber housing having a spring member positioned on top of the outer walls 85. The sensor housing 84 is dimensioned to cover the monitoring sensor 82 and sits inside the sensor receiving wall 46 However, it is possible in the alternative, that the sensor housing 84 wrap around the sensor receiving wall 46. In the embodiment shown, the outer walls 85 fit closely with an inner surface of the sensor receiving wall 46, and the spring member 86 sits between the outer walls 85 and the base 36. By pressing the sensor housing 84 downward, toward the monitoring sensor 82, the power switch 80 is activated.

Now with reference to the drawings, assembly of the major components for the health monitoring apparatus 1 shown in FIGS. 1 and 2 will be discussed.

Firstly, the body section 30 is assembled with the PCB 32 and the flow meter section 34. In particular, the axle 60 is positioned through the base 36, i.e. through a through-hole, and extends upward into a center of the rotor receiving section 50. The axle base 61 is then positioned on an upper surface of the PCB 32, such that the axle base sits between the lower surface of the base 36 in the embodiment shown. The axle base 61 is secured to the PCB 32 and conductive pathways link the rotor movement detector 90 to the CPU IC.

The rotor 55 is the positioned between the pair of channel walls 38 and in direct path of the fluid flow channel 44. In particular, when the rotor 55 is positioned in the rotor receiving section 50, the hub 54 receives the axle 60 and the rotor 55 rests just above the base 36. The plurality of rotor blades 56 extend outward and sits substantially flush with the pair of channel walls 38. The rotor 55 is free to rotate around the axle 60 in the rotor receiving section 50 without the rotor blades 56 touching the channel walls 38.

The communication device 92 extends through a notch in the base 36 and is positioned adjacent to one of the pair of battery receiving sections 62.

The power switch 80 is positioned to extend through the base 36 and into the sensor receiving wall 46. The monitoring sensor 82 is then positioned adjacent to the power switch 80 and, in the embodiment shown, the monitoring sensors 82 rests on top of the control mechanism of the power switch 80. The sensor housing 84 is then positioned in the sensor receiving wall 46, and seals the power switch 80 and the monitoring sensor 82 within the receiving passageway between the sensor receiving wall 46.

In order to secure the PCB 32 with the flow meter section 34, a plurality of fasteners are used. The fasteners, such as screws, are positioned through the base 36 and attached to PCB 32, in the embodiment shown. However, one skilled in the art should appreciate that other connection mechanisms are possible, including adhesives, bolts and nuts, and molding techniques.

In the shown embodiment, the pair of battery receiving sections 62 are the electrically connected to the PCB 32 using wires. As a result, when the power source is positioned in each of the battery receiving section 62, the PCB 32 and components connected to the PCB 32 are powered. As shown in FIGS. 1 and 2, a pair of batteries are positioned in the battery receiving sections 62 with the terminals touching and electrically connected with the metal plates 66.

In the embodiment shown, the mouthpiece housing 74 is removably attached to the flow meter section 34. As a result, the mouthpiece housing 74 is positioned around the first and second mouth probes 70, 72 such that the mouthpiece housing 74 snugly fits around the first and second mouth probes 70, 72 and flush against the base 36 and pair of channel walls 38.

Next, the housing section 10 is assembled over the PCB 32 and the flow meter section 34, such that the upper housing 12 covers the flow meter section 34 and the lower housing 18 covers the PCB 32. The fasteners, such as screws, are positioned through the plurality of fastener receiving through holes in upper housing 12, further extend through body section 30, and secure with fastener receiving through holes in the lower housing 18. The upper and lower housing 12, 18 seamlessly attach to each other in the embodiment shown. The upper and lower housings 12, 18 includes openings along their respective outer walls 14, 20, in order to expose components of the body section 30. The communications device receiving passageway 15, 21 exposes the communication device 92, the fluid entrance passageway 16, 22 exposes the fluid receiving passageway 48 leading into fluid flow channel 44. Furthermore, the fluid entrance passageway 16, 22 is dimensioned to receive the mouthpiece housing 74, as well as the first and second mouth probes 70, 72 that extend there through. The fluid exhaust passageway 17, 23 exposes the fluid exhaust passageway 52

One skilled in the art should appreciate that the upper and lower housings 12, 18 could be attached to each other using a variety of known connection mechanisms, including welding, adhesives, fasteners, or molding techniques.

Lastly, the cover 24 is placed over the mouthpiece section 68.

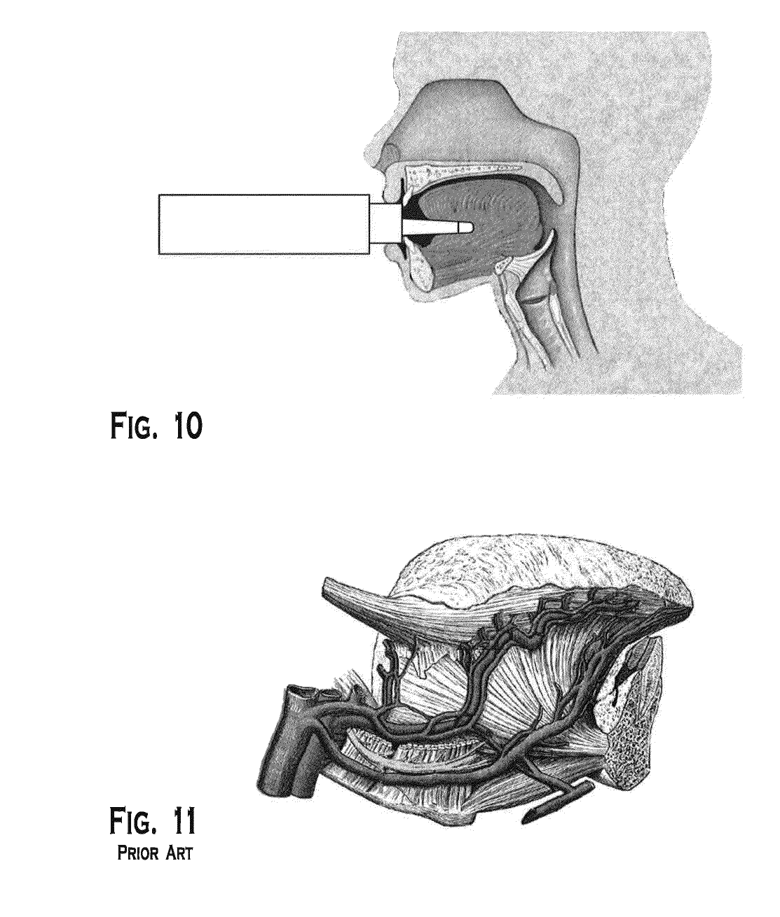

Now with reference to FIGS. 1, 2, 10, and 11 an exemplary method of use for the health monitoring apparatus 1 will be discussed.

In general, the health monitoring apparatus 1 is disposed alongside an individual's bedside near or next to a waking device (e.g., alarm clock). Upon waking, PFW, Tw, and heart rate are acquired and run through an algorithm, which assists in early detection of a developing infection, respiratory event, and/or respiratory infection.

In particular, the mouthpiece section 68 is placed in the user's mouth. The mouthpiece housing 74 is configured to be placed in the mouth and a user's lips form a fluid tight seal around the mouthpiece housing 74. The first and second mouth probes 70, 72 are positioned in the mouth such that the leading ends of the first and second mouth probes 70, 72 are positioned respectively on each side of the tongue, nearest the sublingual artery A where core temperature can best be measured.

The user then presses down on the sensor housing 84. The sensor housing 84 pushes down against the monitoring sensor 82, which then pushes down on the control mechanism for the power switch 80. The control mechanism then activates the health monitoring apparatus 1, by supplying power from the batteries to the PCB 32 and other connected components, including the data collecting components.

Once power is supplied to the health monitoring apparatus 1, data can be collected from the patient and stored in the memory, which is then used in an algorithm to determine the likelihood of a developing infection, respiratory event and respiratory infection, differentiating between the three.

In order to measure heart rate, the user maintains finger position on the sensor housing 84, and the monitoring sensor 82 quickly measures a heart rate of the user, as well as other vitals. In fact, in the shown embodiment, the heart rate is automatically determined after the user activates the power witch 80. While the health monitoring apparatus is powered, the user positions his/her finger on the plastic housing until a heart rate is determined. The heart rate is then collected and stored into the memory.

In order to determine the PFW, the user performs an exhalation into the health monitoring apparatus 1, by directing exhaled fluid through the fluid receiving passageway 48 and into the fluid flow channel 44 to measure PFW. The exhaled fluid is directed toward the rotor receiving section 50 and, in particular, the exhaled fluid is focused by the converging pair of channel walls 38. Then, the exhaled fluid enters the rotor receiving section 50 and rotates the rotor blades 56 about the axle 60. The magnet device 58 turns as the rotor 55 turns, and the rotor movement detector 90 senses the rotating magnet device 58 within the rotor 55. In the embodiment shown, the PFW is determined by counting the number of rotations of the rotor 55 per second. The collected PFW is stored in the memory.

The Tw is then measured by the thermometer tip 78 because the first mouth probe 70 is positioned against the tongue. In particular, the thermometer tip 78 is positioned against the sublingual artery A to measure basal metabolic temperature and provide a digital value of the Tw. The collected Tw is then stored in the memory.

Accordingly, within a few seconds the health monitoring apparatus 1 identifies the user's heart rate, PFW, Tw. This collected data is then used in an algorithm to monitor the user's health conditions. The data can either be transmitted to and analyzed by an external monitoring system using the communications device 92 (explained later) or analyzed by the CPU IC, both using an embedded algorithm that compares collected data against known normal ranges or baselines.

For instance, the collected Tw temperature is compared by the algorithm to an established “normal” range for waking temperature, which was established from data taken from other users, i.e. over 18,000 data points. The normal ranges would be different between genders. The collected PFW reading may be compared by the algorithm using industry established ranges for peak-flow readings.

Now with reference to FIGS. 3 through 9, other exemplary embodiments of the health monitoring apparatus 1 are shown and include additional data collecting components. For the sake of brevity, like parts of the housing section 10, cover 24, and the body section 30, as previously discussed and shown in FIGS. 1 and 2, will be omitted and only those features that are distinguished from the embodiment shown in FIGS. 1 and 2 will be described.

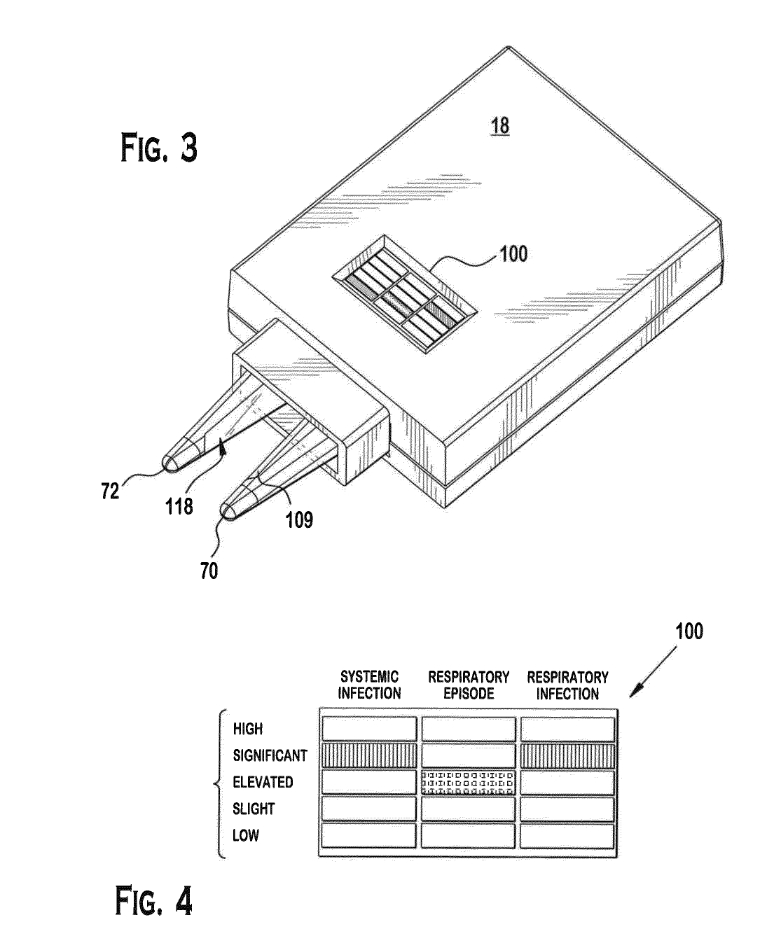

First with reference to FIGS. 3 and 4, the housing section 10 includes an output display 100 disposed along a major surface of the housing section 10. In particular, the output display 100 is disposed along the planar support of the lower housing 18, such that it is easily visible for a user. The output display 100 may be an LCD, LED or OLED display or other suitable type. The output display 100 is a graphical presentation of the user's current health status according to the collected data, and which may be a graph or a bar chart. For instance, the bar chart may include a grid having rows and columns that provide blocks having different colors when illuminated, or other graphical representations known to one skilled in the art.

As shown in FIG. 4, the output display 100 includes rows that indicate the level of probability of an infection or a condition, while the columns designate the general type of infection (i.e. non-respiratory or respiratory) and/or a respiratory condition present. In the embodiment shown, the range of probability includes the following: high, significant, elevated, slight, and low, in descending order. However, one skilled in the art should appreciate that various types of infections, maladies, and episodes can be reported through output display 100, which may vary in shape, dimensions, type and display details depending on the type of data collecting components used with the health monitoring apparatus 1.

According to the invention, the collected data is then run through an algorithm, performed by the CPU IC or an external monitoring system, which determines the level of probability of an infection or an episode, as well as the type of infection and/or episodes present. In the embodiment shown, the output display 100 then indicates the levels of probability for each of the type of infections or episodes, through colored squares or other visual indicator known to one skilled in the art.

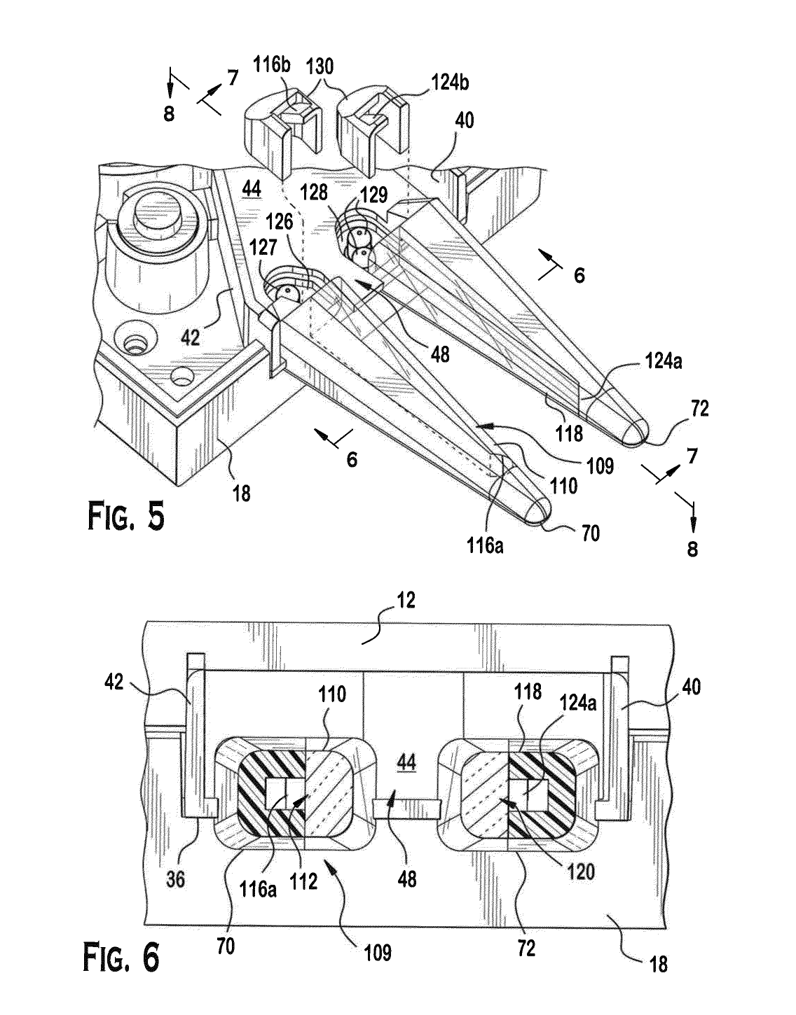

In another exemplary embodiment of the invention, as more particularly shown in FIGS. 5-9, another means to acquire T_{w} is provided.

For instance, as shown in FIGS. 5-8, the mouthpiece section 68 includes a non-contact thermometer light guide 109 disposed along the first mouth probe 70.

In the embodiment shown, the non-contact thermometer light guide 109 includes a first light guide 110 positioned along an inner surface of the first mouth probe 70. In particular, the first light guide 110 includes a first front reflector 116a which is capable of receiving infrared from the surface of the tongue, passing the infrared light through and a first light transmitting passageway 112 that extends through to a first sensor cap housing 130 which includes a light receiving recess 126 and a first rear reflector 116b, which directs the light down ward to a electromagnetic radiation sensor 127.

As shown in FIGS. 5, 7 and 8, the base 36 includes a light receiving recess 126 positioned along a trailing end of the first light guide 110. In the embodiment shown, the PCB 32 includes a electromagnetic radiation sensor 127, such as a photo detector, positioned on the PCB 32 and electrically connected to the CPU IC and memory. The infrared light received by the electromagnetic radiation sensor 127 is converted by the CPU IC to a temperature value and stored in the memory unit.

In the shown embodiment, the first and second mouth probes 70, 72 are disposed axially with respect to the mouthpiece section 68, so as to locate the leading ends (i.e. tips) of the first and second mouth probes 70, 72 accurately in every plane on each side of the tongue nearest the sublingual artery A, where core temperature can best be measured.

Accordingly, in use, the non-contact thermometer light guide 109 directs infrared radiation from the vertical muscle of the tongue through the first light guide 110 and to the electromagnetic radiation sensor 127 using the first front reflector 116a and a first rear reflector 116b in the light receiving recess 126. The electromagnetic radiation sensor 127, which is positioned at the trailing end of the first light guide 110 senses the temperature (Tw) of the tongue muscle by means of related circuitry. It is also possible to detect a heart rate (FIR) using the same technique.

In another exemplary embodiment of the invention, as shown in FIGS. 5-8, a blood oxygen saturation detector 117 is provided.

The blood oxygen saturation detector 117 uses the components of the non-contact thermometer light guide 109, as well as a second light guide 118 positioned along an inner surface of the second mouth probe 72.

In particular, the second light guide 118 includes a second light transmitting passageway 120 that extends from a second rear reflector 124b in a second sensor cap housing 130 to a second front reflector 124a near the end of the second mouth probe 72. The second front reflector 124a is angled toward the first mouth probe 70 and the first front reflector 116a along a leading end of the second light guide 118.

The first and second front reflectors 116a, 124a, as well as the first and second rear reflectors 116b, 124b may be a reflective material, such as foil, or a mirror.

As shown in FIGS. 5, 7 and 8, the base 36 further includes a light directing recess 128 is positioned at a trailing end of the second light guide 118.

In the embodiment shown, the PCB 32 includes one or more light sources 129, such as a 660 nm red LED or 940 nm infrared LED, also positioned on the PCB 32 and electrically connected to the CPU IC and memory. The one or more light sources 129 is positionable with the light directing recess 128. Therefore, in the embodiment shown, when the base 36 and the PCB 32 are secured together, the electromagnetic radiation sensor 127 is positioned in the light receiving recess 126, while the one or more light sources 129 is positioned in the light directing recess 128. Furthermore, a first and second sensor cap housing 130 is provided on top of the light receiving recess 126 and the light directing recess 128 respectively. The first and second sensor cap housing 130 is opaque, and in the embodiment shown, made from a plastic. The first and second sensor cap housing 130 seals the light receiving recess 126 and the light directing recess 128, such that only light can enter or escape the light receiving recess 126 and the light directing recess 128 through the first and second light guides 110, 118, respectively.

Since the first and second mouth probes 70, 72 are disposed axially with respect to the mouthpiece section 68, and on each side of the frenulum of the tongue, the blood oxygen saturation detector 117 provides a means to detect SpO₂ by directing beams of light from one or more light sources 129 to the electromagnetic radiation sensor 127. In particular, light is directed from the one or more light sources 129 down the second light guide 118, then away from the second light guide 118 using the second front reflector 124a. The light then passes through the vertical muscle of the tongue to the first light guide 110. The light travels through the first light guide 110 and to the electromagnetic radiation sensor 127 using the first front reflector 116a and the first rear reflector 116b in the first sensor cap housing 130. The electromagnetic radiation sensor 127, which is positioned at the trailing end of the first light guide 110 receives the light, and by means of the related circuitry on PCB 32, the blood oxygen saturation percentage of the blood in tongue muscle is determined.

In the embodiment shown, two light sources are used, including an LED emitting a 660 nm wavelength and a 940 nm wavelength, pulsed alternately. The pulsed beams are sensed by the electromagnetic radiation sensor 127, and related circuitry determines the blood oxygen saturation percentage of the blood in tongue muscle.

As shown in FIG. 9, in another exemplary embodiment of the invention, an FEO₂ detector 132 is provided. In particular, the FEO₂ detector is disposed along the angled second channel wall 42. The FEO₂ detector 132 is connected to PCB 32 and by means of related circuitry, senses FEO₂ readings.

In yet another exemplary embodiment of the invention, an FENO detector 134 is also provided. As shown in FIG. 9, the FENO detector is disposed along the angled second channel wall 42. The FENO detector 134 is connected to PCB 32 and, by means of related circuitry, sense both FENO percentage readings.

In another embodiment, the communications device 92 connects to an external monitoring system having a processor to analyze data collected from the health monitoring apparatus 1 and a display unit to display results of an impending infection or an impending respiratory event of a particular user.

In particular, the health monitoring apparatus 1 collects data including, but not limited to waking temperature (Tw), waking peak Flow (PFw), and any or all of the following: respiratory infection analysis upon waking (RIW), waking forced expiratory volume at 1 second (FEV1w), waking heart rate taken at the middle finger (HRdw), waking heart rate taken at the vertical muscle of the tongue (HRsw), blood oxygen saturation (SpO2w), waking fractional exhaled oxygen (FEO2w), waking fractional exhaled nitric oxide (FENOw), and waking blood sugar (BSw), which may be acquired by the health monitoring apparatus 1, which is then forwarded using the communications device 92. In an exemplary embodiment, the CPU IC automatically connects to and sends collected data to the external monitoring system. The external monitoring system has a responding computer server and storage unit to receive and store the collected data. The responding computer server stores the collected data in a database by user. The responding computer server then processes incoming data using an embedded algorithm that compares the collected data against known normal ranges or baselines, in an exemplary embodiment. In another embodiment, the responding computer server uses an algorithm to analyze the collected data by user and determine the likelihood of a developing infection, respiratory event and respiratory infection, differentiating between the three.

The external monitoring system then prepares a displayable output from the analyzed collected data and then makes the collected data and displayable output available to the user. In an exemplary embodiment, the user may access the external monitoring system using an Internet website. For instance, in an embodiment of the invention, the user accesses the collected data and the displayable output using a secured website that provides a general user interface that displays the graph and/or collected data. The displayable output is a graphical presentation of the user's current health status according to the collected data, and which may be a graph or a bar chart. For instance, the graph may be similar to the output display 100 described above or other graphical representations known to one skilled in the art. In another embodiment, the graphical presentation could be a conversational-style narrative analysis developed by the same algorithm. The secured website may be accessible through a computer desktop, a laptop, tablet computers, as well as hand-held wireless devices. No wireless device application software (App) will be needed or required.

The foregoing illustrates some of the possibilities for practicing the invention. Many other embodiments are possible within the scope and spirit of the invention. It is, therefore, intended that the foregoing description be regarded as illustrative rather than limiting, and that the scope of the invention is given by the appended claims together with their full range of equivalents.

What is claimed is:

1. A health monitoring apparatus comprising:
a body section having:
a printed circuit board,
a flow meter section positioned adjacent the printed circuit board and having a base and a pair of channel walls extending upward from the base and having a gap there between to define a fluid flow channel with a fluid receiving passageway;
a first mouth probe extending outward from the fluid flow channel;
a second mouth probe disposed axially with respect to the first mouth probe and extending outward from the fluid flow channel; and
a non-contact thermometer light guide disposed along the first mouth probe and leading into the flow meter section and having an electromagnetic radiation sensor along a trailing end of the non-contact thermometer light guide; and
a housing section covering the body section.

2. The health monitoring apparatus according to claim 1, wherein the flow meter section further includes a waking peak flow data collecting component positioned in the fluid meter section.

3. The health monitoring apparatus according to claim 1, wherein the pair of channel walls include a first channel wall that extends linearly from a front side of the base toward a substantial central section of the base and a second channel wall that extends from the front side toward the rear at an angle toward the first channel wall.

4. The health monitoring apparatus according to claim 3, wherein the fluid flow channel leads into a rotor receiving section positioned between the first channel wall and the second channel wall.

5. The health monitoring apparatus according to claim 4, wherein the rotor receiving section is circular shaped with the pair of channel walls mirroring the other around a proximate central section of the base.

6. The health monitoring apparatus according to claim 4, wherein the flow meter section further includes a waking peak flow data collecting component positioned in the rotor receiving section.

7. The health monitoring apparatus according to claim 6, wherein the waking peak flow data collecting component includes a rotor positioned and rotatable in the rotor receiving section.

8. The health monitoring apparatus according to claim 7, wherein the rotor includes a hub and a plurality of rotor blades extending outward from the hub and in direct path of the fluid flow channel.

9. The health monitoring apparatus according to claim 8, wherein the rotor includes a magnetic device embedded therein.

10. The health monitoring apparatus according to claim 9, further comprising a rotor movement detector disposed in the body section and corresponding in position with a travel path of the magnetic device as the rotor rotates in the rotor receiving section.

11. The health monitoring apparatus according to claim 1, wherein first and second mouth probes are elongated members having respective trailing ends positioned through the fluid receiving passageway and secured to an interior upper surface of the base.

12. The health monitoring apparatus according to claim 11, further comprising a mouthpiece housing having outer walls and a mouth probe receiving passageway extending between the outer walls such that the outer walls wrap around each of the first and second mouth probes and abuts a front edge of the base.

13. The health monitoring apparatus according to claim 12, wherein the body section is shaped to form a seal with a user's lips in order to block ambient light.

14. The health monitoring apparatus according to claim 1, further comprising a monitoring sensor positioned in a sensor receiving wall extending upward from the base and through the housing section.

15. The health monitoring apparatus according to claim 14, wherein the monitoring sensor is an IR sensor capable of determining heart rate.

16. The health monitoring apparatus according to claim 15, further comprising a power switch positioned on the printed circuit board and leading into the sensor receiving wall.

17. The health monitoring apparatus according to claim 16, wherein the monitoring sensor is positioned adjacent to the power switch and covered by a sensor housing with a spring member.

18. The health monitoring apparatus according to claim 1, wherein the non-contact thermometer light guide includes a first light guide positioned along an inner surface of the first mouth probe and leading into the flow meter section.

19. The health monitoring apparatus according to claim 18, wherein the electromagnetic radiation sensor is positioned at a trailing end of the first light guide and in the fluid flow channel.

20. The health monitoring apparatus according to claim 19, wherein the first light guide includes a first light transmitting passageway that extends through an interior of the first mouth probe and is capable of passing light there through and down the first light transmitting passageway.

21. The health monitoring apparatus according to claim 20, wherein the first light guide further includes a first front reflector positioned along the first mouth probe to direct light down the first light transmitting passageway.

22. The health monitoring apparatus according to claim 21, wherein the first front reflector is an angled mirror directed at the electromagnetic radiation sensor.

23. The health monitoring apparatus according to claim 22, wherein the base includes a light receiving recess positioned along the trailing end of the first light guide and receiving the electromagnetic radiation sensor.

24. The health monitoring apparatus according to claim 23, wherein the electromagnetic radiation sensor is a photo detector and covered by a sensor cap housing disposed over the light receiving recess.

25. The health monitoring apparatus according to claim 24, wherein the first light guide directs infrared radiation from a vertical muscle of a user's tongue to the electromagnetic radiation sensor using the first front reflector.

26. The health monitoring apparatus according to claim 24, further comprising a second light guide disposed along an inner surface of the second mouth probe and facing the first light guide.

27. The health monitoring apparatus according to claim 26, wherein the second light guide includes a second light transmitting passageway that extends through an interior of the second mouth probe capable of passing light there through and down the second light transmitting passageway.

28. The health monitoring apparatus according to claim 27, wherein the second light guide includes a second front reflector to assist in directing light down the second light transmitting passageway.

29. The health monitoring apparatus according to claim 28, wherein the second front reflector is an angled mirror directed to the first front reflector directing light through a vertical muscle of a user's tongue.

30. The health monitoring apparatus according to claim 29, wherein the base includes a light directing recess positioned along the trailing end of the second light guide and receiving a light source.

31. The health monitoring apparatus according to claim 30, wherein the light source includes a 660 nm red LED and 940 nm infrared LED.

32. The health monitoring apparatus according to claim 1, wherein the pair of channel walls includes an angled wall extending into the fluid flow channel.

33. The health monitoring apparatus according to claim 32, further comprising a FEO₂ detector positioned along the angled wall.

34. The health monitoring apparatus according to claim 33, further comprising a FENO detector positioned along the angled wall.

35. The health monitoring apparatus according to claim 32, further comprising a FENO detector positioned along the angled wall.

36. The health monitoring apparatus according to claim 1, further comprising a communication device disposed in the body section and accessible through the housing section.

37. The health monitoring apparatus according to claim 36, wherein the communication device is a modular connector socket.

38. The health monitoring apparatus according to claim 1, further comprising an output display disposed along a major surface of the housing section.

39. The health monitoring apparatus according to claim 38, wherein the output display is a visual screen having an indicator for a probability of a present infection.

40. The health monitoring apparatus according to claim 39, wherein the visual screen includes a grid with rows and columns.

41. A health monitoring apparatus comprising:
a body section having:
a flow meter section having fluid flow channel with a waking peak flow data collecting component positioned therein;
a mouthpiece section shaped having a pair of mouth probes disposed axially with respect to each other and a waking body temperature data collecting component disposed on one of the pair of mouth probes; and
a printed circuit board positioned adjacent and connected to the flow meter section and having a processing unit with an algorithm to analyze data collected by the waking peak flow data component and the waking body temperature data component in order to display a result of an impending infection or an impending respiratory event; and
a housing section having a display system to display the result.

42. The health monitoring apparatus according to claim 41, wherein the flow meter section includes a base and a pair of channel walls extending upward from the base and having a gap there between to define the fluid flow channel having a fluid receiving passageway.

43. The health monitoring apparatus according to claim 42, wherein the pair of channel walls include a first channel wall that extends linearly from a front side of the base toward a substantial central section of the base and a second channel wall that extends from the front side toward the rear at an angle toward the first channel wall.

44. The health monitoring apparatus according to claim 43, wherein the fluid flow channel leads into a rotor receiving section positioned between the first channel wall and the second channel wall.

45. The health monitoring apparatus according to claim 44, wherein the rotor receiving section is circular shaped with the pair of channel walls mirroring the other around a proximate central section of the base.

46. The health monitoring apparatus according to claim 45, wherein the waking peak flow data collecting component is positioned in the rotor receiving section.

47. The health monitoring apparatus according to claim 46, wherein the waking peak flow data collecting component includes a rotor that is rotatable in the rotor receiving section.

48. The health monitoring apparatus according to claim 47, wherein the rotor includes a hub and a plurality of rotor blades extending outward from the hub and in direct path of the fluid flow channel.

49. The health monitoring apparatus according to claim 48, wherein the rotor includes a magnetic device embedded therein.

50. The health monitoring apparatus according to claim 49, further comprising a rotor movement detector disposed in the body section and corresponding in position with a travel path of the magnetic device as it rotates in the rotor receiving section.

51. The health monitoring apparatus according to claim 41, wherein the waking body temperature data collecting component is a thermometer.

52. The health monitoring apparatus according to claim 41, wherein the waking body temperature data collecting component is a non-contact thermometer light guide having a first light guide positioned along an inner surface of one of the pair of mouth probes and leading into the flow meter section.

53. The health monitoring apparatus according to claim 52, further comprising an electromagnetic radiation sensor positioned at a trailing end of the first light guide and in the fluid flow channel.

54. The health monitoring apparatus according to claim 53, wherein the first light guide includes a first light transmitting passageway that extends through an interior of the one of the pair of mouth probes and is capable of passing light there through and down the first light transmitting passageway.

55. The health monitoring apparatus according to claim 54, wherein the first light guide further includes a first front reflector positioned to direct light down the first light transmitting passageway.

56. The health monitoring apparatus according to claim 55, wherein the first front reflector is an angled mirror directed to the electromagnetic radiation sensor.

57. The health monitoring apparatus according to claim 56, wherein the base includes a light receiving recess positioned along the trailing end of the first light guide and receiving the electromagnetic radiation sensor.

58. The health monitoring apparatus according to claim 57, wherein the electromagnetic radiation sensor is a photo detector and covered by a sensor cap housing disposed over the light receiving recess.

59. The health monitoring apparatus according to claim 58, wherein the first light guide directs infrared radiation from a vertical muscle of a user's tongue to the electromagnetic radiation sensor using the first front reflector.

60. The health monitoring apparatus according to claim 41, wherein the display system is a visual screen having a grid with rows and columns that provide an indicator for a probability of a present infection.

61. A health monitoring system, comprising:
a health monitoring apparatus having:
a flow meter section having fluid flow channel with a waking peak flow data collecting component positioned therein;
a mouthpiece section shaped having a pair of mouth probes disposed axially with respect to each other and a waking body temperature data collecting component disposed on one of the pair of mouth probes;
a printed circuit board positioned adjacent and connected to the flow meter section and having a processing unit to process data collected by the waking peak flow data component and the waking body temperature data component and a communication device; and
an external monitoring system connected to the communication device and capable of receiving, storing and analyzing collected data in a storage device, the remote computer system having a processor to analyze data collected and a display unit to display a result of an impending infection or an impending respiratory event of a particular user of the health monitoring apparatus.
